(19) United States
(12) Patent Application Publication
(10) Pub. No.: US 2005/0202085 A1
(43) Pub. Date: Sep. 15, 2005

(54) Unit dose of material in system and method

(76) Inventor: Dale R. Lovercheck, Media, PA (US)

Correspondence Address: Dale R. Lovercheck, 92 Patricia Place, Media, PA, 19063, US

(21) Appl. No.: 11/080,733

(22) Filed: Mar. 15, 2005

Related U.S. Application Data
(63) Continuation of application No. 09/900,647, filed on Jul. 7, 2001.
(60) Provisional application No. 60/216,924, filed on Jul. 8, 2000.

(51) Int. Cl. A61K 31/522; A61K 31/192; A61K 33/24

(52) U.S. Cl. 424/464; 514/263.31; 514/569; 424/617

(57) ABSTRACT

The invention provides a unit dose of an orally consumable material, having a predetermined pharmaceutically effective amount of at least one nonprescription discomfort reliever and a predetermined nutritionally effective amount of at least one nutritional supplement. Each unit dose may be in a container having indications of the amount discomfort reliever and the amount of nutritional supplement in each unit dose. Instructions are provided for consuming the material for discomfort relief and supplementing nutrition. Consumption of the unit dose simultaneously relieves discomfort and supplements nutrition.

This application claims the benefit of U.S. Provisional patent application No. 60/216,924 filed Jul. 8, 2000, which is incorporated herein by reference in its entirety.

The invention relates to unit (single) doses of orally consumable material. More specifically, the invention provides unit doses of orally consumable material for both discomfort relief and supplementING nutrition. The invention is particularly adapted for consumers having persistent discomfort who are regularly in need of relief. The more persistent the discomfort the more regularly consumers supplement their nutrition while relieving their discomfort in accordance with the invention.

Beneficially consumers are able to regulate and monitor their consumption of nutritional supplements with their consumption of discomfort relievers. Beneficially, the invention omits one of each two containers required by the prior art. Consumers of unit dose tablets of the invention purchase, carry, store and open half as many containers and to consume half as many tablets. Yet they obtain the same pharmaceutically and nutritionally effective benefits as the prior art.

### BACKGROUND OF THE INVENTION

The evidence indicates that for many people, it makes good sense to use nutritional supplements regularly, as see COUNCIL FOR RESPONSIBLE NUTRITION, The Benefits of Nutritional Supplements, Executive summary, 2001. Nonprescription orally consumable materials for discomfort relief are made and sold separately from orally consumable materials for nutritional supplements. These discomfort relievers and nutritional supplements require separate manufacturing, packaging, and advertising. Large portions of storage space are required to store discomfort relievers and nutritional supplements, both by wholesalers and retailers for distribution and by consumers for availability for their use. The more available for use the consumer keeps discomfort relievers and nutritional supplements, the greater the amount of expiration waste and the greater the amount of time required for restocking. Consumers typically lack regularity in consumption of nutritional supplements due to lack of immediate access to them at times when consumption is desired or due to failure to remember to consume them. It is often difficult for consumers to monitor the amounts of their consumption of nutritional supplements due to their irregular consumption, for example in food products, such as calcium nutritional supplement in orange juice. A further problem of the prior art is the large volume (size) of some nutritional supplement tablets, such as 500 mg (1000 mg) Vitamin C tablets. Nutritional supplement or discomfort reliever is disclosed in U.S. Pat. No. 4,820,524; U.S. Pat. No. 5,087,454; U.S. Pat. Nos. 4,664,915; 5,436,026 and U.S. Pat. No. 5,587,363 each of which is incorporated herein by reference in its entirety. The problems of the prior art are overcome by the present invention.

An advantage for consumers of combined tablets of the invention over separate tablets is supplementing nutrition when consuming discomfort reliever. The combined tablets improve nutrition through more consistent consumption of nutritional supplements. combined tablets provide smaller tablet size. They reduce waste, cost and storage space of nutritional supplements.

Also, the discomfort reliever may be uniformly distributed in the nutritional supplement to reduce the mass of discomfort reliever per unit volume of orally consumable material and thus passively reduce side effects of some discomfort relievers. The advantages of the invention over separate tablets for manufacturers include: a substantial reduction in cost of manufacture for containers, labels and inactive ingredients and a substantial reduction in cost of storage and shipping.

### SUMMARY OF THE INVENTION

The invention provides a unit dose of an orally consumable material, having a predetermined pharmaceutically effective amount of at least one nonprescription discomfort reliever and a predetermined nutritionally effective amount of at least one nutritional supplement. Each unit dose may be in a container having indications of the amount discomfort reliever and the amount of nutritional supplement in each unit dose. Instructions are indicated for consuming the material for discomfort relief and supplementing nutrition. Consumption of the unit dose simultaneously relieves discomfort and supplements nutrition.

### DETAILED DESCRIPTION OF THE INVENTION

A unit dose of an orally consumable material of the invention has a predetermined pharmaceutically effective amount of at least one nonprescription discomfort reliever and a predetermined nutritionally effective amount of at least one nutritional supplement. “Orally consumable material” as used herein, refers to material adapted to be safely consumed orally by a person. Orally consumable material, as used herein, does not include material adapted to be taken intravenously (by injection into a vein) or nasally (by injection into the nasal passages), or adapted for topical application, for example by applying to the outer surface of the skin. Orally consumable material which includes discomfort reliever(s) and nutritional supplement(s) in accordance with the invention is preferably stored in solid form, such as a powder (for example in a capsule) or powder compressed into a unit dose tablet. A unit dose is a unit to be consumed as a single dose. The orally consumable material may, for example, include caramel, chocolate, or chewing gum.

“Discomfort” as used herein, refers to discomfort from at least one of: minor aches and pain associated with a common cold, headache, toothache, backache, muscular aches, menstrual cramps, minor pain of arthritis, fever, running nose, sneezing, itching of nose or throat, itchy watering eyes due to hay fever or other upper respiratory allergy, insomnia (difficulty in falling asleep), sleepiness, fatigue and drowsiness.

“Discomfort reliever” as used herein, refers to predetermined pharmaceutically effective amounts of orally consumable material adapted for temporary relief of at least one predetermined discomfort. Such amounts of this material are widely used and widely recognized as being safe and effective for temporary relief of at least one predetermined discomfort. Exemplary of a prior art discomfort reliever is a 200 mg ibuprofen tablet sold by Whitehall Robins Healthcare under the trademark Advil (pain reliever), as see U.S. Pat. No. 5,087,454. By contrast, U.S. Pat. No. 5,948,443 discloses acetylsalicylic acid and micronutrient supplementation for nutritional losses and coronary heart disease. This use of acetylsalicylic acid is not as a discomfort reliever, as coronary heart disease is not a discomfort (as used herein). Minor aches and pain associated with a common cold, headache, tooth ache, backache, minor pain of arthritis, are most preferred discomforts relieved by discomfort relievers used in accordance with the invention. Nonprescription pain relievers, nonprescription allergy medication and nonprescription cold medication, analgesics, nasal decongestants, and antihistamines are preferably used as discomfort relievers for relieving discomfort in accordance with the invention. Buffered aspirin with an acid-regulator is a discomfort reliever. Most preferred discomfort relievers are analgesic non-prescription, non-aspirin, non-steroid anti-inflammatory drugs (NSAIDS), such as ibuprofen, naproxen, or ascetaminophen frequently used for temporary relief of pain, fever and/or inflammation. Non-aspirin discomfort reliever is discomfort reliever other than aspirin and precursors of aspirin. Nonprescription discomfort relievers are discomfort relievers that do not require a physician's prescription.

Discomfort relievers include antihistamine. The antihistamine may, for example, include chlorpheniramine maleate, antazoline phosphate, bromodiphenhydramine hydrochloride, brompheniramine maleate, carbinoxamine maleate, chlorcyclizine HCl, chlorothen citrate, clemizole HCl, cromolyn sodium, dexchlorpheniramine maleate, diphenhydramine HCl, diphenylpyraline HCl, doxylamine succinate, methapyrilene fumarate, methapyrilene HCl, methdilazine, methdilazine HCl, phenindamine tartrate, promethazine HCl, pyrilamine maleate, pyrrobutamine phosphate, rotoxamine tartrate, trimeprazine tartrate, tripelennamine citrate, tripelennamine HCl, triprolidine HCl, and chlorpheniramine gluconate.

“Nutritional supplement” as used herein, refers to predetermined nutritionally effective amounts of orally consumable material for supplementing nutrition. Nutritional supplements include predetermined amounts of orally consumable vitamin(s), mineral(s) and/or herb(s) as supplement(s) adapted for nutrition. Nutritionally effective amounts, include amounts of orally consumable material widely used for supplementing nutrition and amounts of orally consumable material widely recognized as being safe and effective for supplementing nutrition. Preferred nutritional supplements are vitamin(s) and/or mineral(s) most commonly sold without prescription for supplementing nutrition. Commercially available unit dose multiple vitamin and mineral products are nutritional supplements having predetermined nutritionally effective amounts of orally consumable material for supplementing nutrition. Such unit dose products are commercially available and sold for example under the trademarks: Centrum Silver (with lutein iron free formula), Theragran Heart Right and One A Day. By contrast, U.S. Pat. No. 5,128,334 discloses a method for reducing the side effects of aspirin in a mammal with ascorbic acid and derivatives of ascorbic acid. In this pain reliever containing aspirin, the ascorbic acid and derivatives of ascorbic acid are not nutritional supplements, as they are not predetermined amounts adapted (indicated or functional) for supplementing nutrition. Preferred multiple vitamin and mineral nutritional supplements include at least four members of the group consisting of: vitamin A, vitamin C (ascorbic acid), vitamin D, vitamin E, vitamin K, vitamin B₁₂, vitamin B₆, thiamin, riboflavin, niacin, foliate, biotin, pantothenic acid, calcium, iron, phosphorous, iodine, magnesium, zinc, selenium, copper, manganese, chromium, molybdenum, chlorine, or potassium nickel, tin, silicon, vanadium, boron.

Each unit dose may be in a container having indications of the amount discomfort reliever and the amount of nutritional supplement in each unit dose. Instructions are provided for consuming the material for discomfort relief and supplementing nutrition. Consumption of the unit dose simultaneously relieves discomfort and supplements nutrition.

A unit dose of an orally consumable material in accordance with a preferred embodiment of the invention has an indicated discomfort reliever and an indicated nutritional supplement. “Indicated discomfort reliever” as used herein, refers to discomfort reliever in a system having an indicator indicating a pharmaceutically effective amount of the discomfort reliever per unit dose for relieving discomfort. The system may for example, include a container containing the unit dose and supporting the indicator, such as a printed paper or label. For example, a 200 mg ibuprofen tablet sold by Whitehall Robins Healthcare under the trademark Advil (pain reliever) packaged in a container having a label indicating a pharmaceutically effective amount of the discomfort reliever per unit dose (per tablet) for relieving indicated discomforts is an indicated discomfort reliever. By contrast, U.S. Pat. No. 5,948,443 discloses acetylsalicylic acid and micronutrient supplementation for nutritional losses and coronary heart disease, the acetylsalicylic acid is not an indicated discomfort reliever as it is not a predetermined amount of discomfort reliever indicated for relieving discomfort. Preferably an indicated discomfort reliever is not indicated for a purpose other than discomfort relief.

“Indicated nutritional supplement” as used herein, refers to nutritional supplement in a system having an indicator indicating a nutritionally effective amount of the nutritional supplement per unit dose for supplementing nutrition. The system may for example, include a container, such as a plastic bottle having twist off cap and containing one or more tablets each having a nutritionally effective unit dose of nutritional supplement. The container may support an indicator, such as a printed paper or a label. A commercially available Indicated nutritional supplement product is sold by Whitehall Robins Healthcare under the trademark Centrum Silver. These multiple vitamin & mineral tablets are packaged in a container having a label indicating a nutritionally effective amount of each vitamin and mineral per unit dose (per tablet). Preferably an indicated nutritional supplement is not indicated for a purpose other than supplementing nutrition.

A unit dose of an orally consumable material in accordance with a preferred embodiment of the invention has a functional discomfort reliever and a functional nutritional supplement. “Functional nutritional supplement” as used herein, refers to predetermined amounts of nutritional supplement which is initially and/or primarily adapted to function in supplementing nutrition when consumed in orally consumable material containing a discomfort reliever. Functional nutritional supplements are not initially and/or primarily adapted to actively aid in, and/or actively contribute to discomfort relieving functions of a discomfort reliever contained in the orally consumable material. Nor are functional nutritional supplements initially and/or primarily adapted to actively aid in, and/or actively contribute to reducing side effects of a discomfort reliever contained in the orally consumable material. A functional nutritional supplement does not include amounts of nutritional supplements which are therapeutic for either the discomfort, or the cause of the discomfort. For example, when a tablet of pain reliever/fever reducer is combined with a tablet of multiple vitamins & minerals, as in Example 18 below, the predetermined amounts of multiple vitamins & minerals are functional nutritional supplements which upon consumption are initially adapted to function in supplementing nutrition. By contrast, U.S. Pat. No. 5,128,334 discloses a method for reducing the side effects of aspirin in a mammal with ascorbic acid and derivatives of ascorbic acid. In this pain reliever containing aspirin, the ascorbic acid and derivatives of ascorbic acid are not functional nutritional supplements, as they have an excluded function, and are not predetermined amounts of nutritional supplements initially and/or primarily adapted to function in supplementing nutrition. Also, U.S. Pat. No. 6,068,999 discloses a migraine headache symptom reducer including feverfew, magnesium and riboflavin. In this headache symptom reducer magnesium and riboflavin have an excluded function, and are not functional nutritional supplements, as they are not predetermined amounts of nutritional supplements initially and/or primarily adapted to function in supplementing nutrition.

“Functional discomfort reliever” as used herein, refers to predetermined amounts of discomfort reliever which are initially and/or primarily adapted to function in temporarily relieving discomfort when consumed in orally consumable material containing a nutritional supplement. Functional discomfort reliever is not initially and/or primarily adapted to actively aid in, and/or actively contribute to nutrition supplementing functions of a nutritional supplement contained in the orally consumable material. For example, when a tablet of multiple vitamins & minerals is combined with a tablet of pain reliever as in Example 18 below, then the predetermined amount of ibuprofen is a functional discomfort reliever which, upon consumption, is initially adapted to function in temporarily relieving a discomfort. By contrast, in U.S. Pat. No. 5,948,443 which discloses acetylsalicylic acid and micronutrient supplementation for nutritional losses and coronary heart disease, the acetylsalicylic acid is not a functional discomfort reliever as it is not a predetermined amount of discomfort reliever initially and/or primarily adapted to function in relieving discomfort.

Orally consumable material in accordance with the invention may be enclosed in a container having a twist-off cap. The container encloses a plurality of orally consumable tablets. A label may be adhered to the container which has printed thereon indications of the amount of discomfort reliever per tablet and indications of the percent of a daily value (and/or amount) of one or more nutritional supplement(s) per tablet. Preferably the label has printed thereon pharmaceutically effective amounts per tablet of discomfort reliever(s) and nutritionally effective amounts per tablet of nutritional supplement.

Preferred discomfort reliever is an analgesic, for example, a non-aspirin analgesic and the nutritional supplement may be a vitamin, such as vitamin C or a multi-vitamin and mineral supplement. For example, Orally consumable chewable tablets are formed, by adding vitamin C in an amount equal to half the mass of ibuprofen, but otherwise following the procedure of Example I of U.S. Pat. No. 5,320,855, (or Example IX of U.S. Pat. No. 5,215,755) each of which is incorporated herein by reference in its entirety.

Each tablet, and capsule of the invention is a unit dose of an orally consumable material. A sheet of tablets is provided in accordance the invention. A plurality of tablets are supported by a molded transparent plastic sheet with a plurality of container wells. An aluminum foil backing is sealed to the molded sheet enclosing a capsule in each container well. Paper sheet label is adhered to backing. The paper label has printed thereon indications of the pharmaceutically effective amount of discomfort reliever per capsule, and indications of the percent of a daily value (and/or amount) of one or more nutritional supplements per capsule. Each tablet includes a discomfort reliever, such as an antihistamine, for example, diphenhydramine hydrochloride, and a nutritional supplement, such as vitamin C and/or calcium.

Each unit solid material (adapted to be leached) of the invention is adapted to form a unit dose of an orally consumable material. A tea bag is provided in accordance with the invention. Paper is stapled to enclose a unit dose of orally consumable material and a portion of solid material adapted to be leached, such as tea leaves. When placed in water, the orally consumable material flows through the paper into the water to form water having a unit does of nutritional supplement and discomfort reliever. A label is affixed tone end of a string. The other end of a string is held by the staple to the paper. The label has indicated thereon indications of the amount of discomfort reliever per unit dose and indications of the percent of a daily value (and/or amount) of one or more nutritional supplements per unit dose.

Another tea bag of the invention includes a purified synthetic analgesic and plant material adapted to be leached, such as tea and/or herb. The analgesic preferably includes a non-steroid analgesic in addition to feverfew. Nutritional supplement is optionally included.

Each unit dose of orally consumable material has a predetermined pharmaceutically effective amount of one nonprescription discomfort reliever and a predetermined nutritionally effective amount of at least one nutritional supplement. For example non-aspirin analgesics are included with vitamins, minerals, and/or herbs in a unit dose.

In a set of containers is a set of orally consumable material formed for example into pills, tablets, capsules, liquids, powders, pieces and/or sticks. A set of liquid orally consumable material is formed by leaching non-aspirin analgesics and vitamins, minerals, and/or herbs, contained in water permeable bags. Preferred orally consumable material for use in accordance with the invention is in solid form, for example compressed powder.

A consumer using two discomfort relievers and two different nutritional supplements in accordance with the invention eliminates two sets of tablets and two containers. An additional two containers are eliminated at each of location they are used. Exemplary locations are at a primary residence, an office, and/or a vacation home, and/or in an automobile, a truck, a boat, travel luggage, a pocket of an article of clothing and/or in a hand bag. Each container at the same location may have a different nutritional supplement such as multiple vitamins and minerals, iron, calcium or vitamin C.

Nutritional supplement active ingredients preferably include one or more vitamins selected from the group consisting of vitamin A, vitamin B₁, vitamin B₂, vitamin B₆, vitamin B₁₂, vitamin C, vitamin D, vitamin E, niacin and folic acid. Preferably one or more of the foregoing vitamins is in an amount corresponding to a percentage value, such as the U.S. Recommended Daily Allowance (RDA) or daily value within the range of 0.1-100%. For nutritional supplements having a maximum recommended amount per day, the amount of such nutritional supplement per unit dose is preferably equal to or less than the maximum recommended amount (of such nutritional supplement per day) divided by the maximum number of unit doses of the discomfort reliever per day. Iron for children has a maximum recommended amount per day.

A preferred embodiment of the invention includes a container having indicated thereon pharmaceutically effective amounts per unit dose (such as tablet or capsule) of discomfort reliever. Preferred discomfort relievers are anti-inflammatory agents, such as aspirin (or antipyretic, such as aconite). The container also has indicated thereon nutritionally effective amounts per unit dose of at least one nutritional supplement such as vitamin C or a multiple vitamins and minerals. The container encloses a plurality of unit doses.

Preferably a container enclosing a unit dose of analgesic and nutritional supplement in accordance with the invention includes an indication of a percent of a daily value (or amount) for each vitamin, mineral and/or herb of the nutritional supplement per unit dose. The container preferably indicates an expiration date for the first to expire of the nutritional supplement and the analgesic and a bar code identifying the product enclosed in the container.

Preferably the unit dose is enclosed within a container having indications for consuming the material to relieve specified discomfort and to supplement nutrition. Preferably the nutritional supplement is a functional nutritional supplement, the discomfort reliever is a functional discomfort reliever. Preferably the discomfort is selected from the group consisting of pain, upper respiratory allergy, and common cold. Preferably the nutritional supplement is selected from the group consisting of vitamin C, calcium, and multiple vitamins and minerals each in a substantial portion of a recommended daily value (or amount).

The period of consumption is preferably indicated on the label. For example consumption may continue for 7 days, 10 days or for an unlimited period.

Two (or more) indications for use are preferably indicated on the label selected from the group consisting of: minor aches and pain associated with the common cold, headache, tooth ache, backache, minor pain of arthritis, the pain of menstrual cramps, reducing fever, pain relief and nutritional supplement. By contrast, indications for use indicated on the label are not selected from the group consisting of atherosclerosis, inhibiting thrombus formation, reducing risk of coronary heart disease and reduction or prevention of oxidation of low density lipoproteins within coronary arterial walls.

Preferably the discomfort reliever is a non-aspirin analgesic, and the nutritional supplement is selected from the group consisting of about 50 to about 1,000 mg vitamin C, about 50 to about 400 mg vitamin B₂, 2 mg to 30 mg of vitamin B₆, 7 mg to 200 mg of vitamin B₁₂, about 50 to about 500 IU vitamin D, about 50 to about 1,200 mg Ca.

Preferably the orally consumable material is in the form of a pill, tablet, capsule, liquid, powder, bag, piece or stick.

A preferred embodiment of the invention provides a unit dose of orally consumable material, consisting essentially of 50 mg to 300 mg of one or more pharmaceutically effective indicated discomfort reliever compounds and a between 50 mg to 1000 mg of indicated nutritionally effective nutritional supplement. The nutritional supplement may for example be vitamin C, calcium and/or multiple vitamins and minerals. The multiple vitamins and minerals may be adapted for consumption once daily by a majority of men and a majority of women.

In a unit dose of orally consumable material within a preferred embodiment of the invention the active ingredients consist essentially of a predetermined pharmaceutically effective amount of at least one nonprescription functional discomfort reliever and a predetermined nutritionally effective amount of at least one functional nutritional supplement. The unit dose is in an enclosure having an indicator with indications for consuming the material for pain relief and nutritional supplement.

A preferred embodiment of the invention provides a system of unit doses, including: a first set of units doses each including a pharmaceutically effectively amount of a first discomfort reliever and a nutritionally effectively amount of a first nutritional supplement, a second set of units doses each including pharmaceutically effectively amount of a the first discomfort reliever and a nutritionally effectively amount of a second nutritional supplement. Preferably the first nutritional supplement is vitamin C or multiple vitamins and minerals and the second nutritional supplement is calcium or vitamin B₆.

A family of sets of unit doses is provided by a preferred embodiment of the invention. A first set of unit doses each include a pharmaceutically effectively amount of a first discomfort reliever and a nutritionally effectively amount of a first nutritional supplement. A second set of unit doses each includes a pharmaceutically effectively amount of a second discomfort reliever and a nutritionally effectively amount of the first nutritional supplement. The first set of unit doses is in a container having at least two indications for use selected from the group consisting of: running nose, sneezing, itching of nose or throat, itchy watering eyes due to hay fever or other upper respiratory allergy. The second set of unit doses is in a container having at least two indications for use selected from the group consisting of: minor aches and pain associated with the common cold, headache, tooth ache, backache, minor pain of arthritis, the pain of menstrual cramps and reducing fever.

Another family of sets of unit doses is provided by another preferred embodiment of the invention. A first set of unit doses each has a first predetermined pharmaceutically effectively amount of a first discomfort reliever and a first predetermined nutritionally effectively amount of a first nutritional supplement. A second set of unit doses each has the first predetermined pharmaceutically effectively amount of the first discomfort reliever and a second predetermined nutritionally effectively amount of the first nutritional supplement. Exemplary of the first nutritional supplement is vitamin C, calcium, vitamin B₆ and a multiple vitamins and minerals. The first amount of a first nutritional supplement may for example be between one and fifty percent of a daily value (or amount). The second amount of a first nutritional supplement may for example be between one and fifty percent more than the first amount of the first nutritional supplement. The first discomfort reliever may for example be between one and fifty percent of a daily maximum amount.

Two sets of unit doses, may include a first set of unit doses each including a first pharmaceutically effectively amount of a first discomfort reliever and a first nutritionally effectively amount of a first nutritional supplement. Each dose of the second set of units doses has a second pharmaceutically effectively amount of the first discomfort reliever and the first amount of the first nutritional supplement. The second amount of a first discomfort reliever is for example between one and fifty percent more than the first amount of the first discomfort reliever.

Alternatively, the second set of units doses each has a second pharmaceutically effectively amount of the first discomfort reliever and a second nutritionally effectively amount of the first nutritional supplement. The second amount of the first nutritional supplement is between one and fifty percent less than the first amount of a first nutritional supplement, and the second amount of the first discomfort reliever is between one and fifty percent more than the first amount of the first discomfort reliever. Each set is enclosed within a separate container. Each container preferably has at least two indications for use selected from the group consisting of: minor aches and pain associated with the common cold, headache, tooth ache, backache, minor pain of arthritis, the pain of menstrual cramps and reducing fever.

The following examples are offered to aid in understanding the invention and are not to be construed as limiting its scope. Unless otherwise indicated, all parts and percentages are by weight.

In each of EXAMPLES 1 through 8 the specific amounts of the specified components are mixed as the only active ingredients (along with inactive ingredients known in the art) by procedures known in the art to form a product mixture. Each product mixture is made three times to form three duplicate samples of the product mixture. One product mixture sample is formed into a tablet, the second product mixture sample is formed into a capsule and the third product mixture sample is mixed as a powder with one gram of tea leaves and enclosed in a water permeable paper bag and the paper is stapled to a string to form a tea bag all by procedures known in the art.

### EXAMPLE 1

### Pain Discomfort Reliever and Vitamin C

200 mg of ibuprofen, and 100 mg of vitamin C are mixed together to form a product mixture.

### EXAMPLE 2

### Allergy Discomfort Reliever and Multiple Vitamins and Minerals

200 mg of ibuprofen, and the following components in the amounts indicated are mixed together to form a product mixture.

| | |
|---|---|
| 1.34 | mg Clemastine Fumarate |
| 75 | mg phenylpropanolamine, |
| 380 | mg Calcium, |
| 400 | I.U. Vitamin D, |
| 600 | I.U. Beta-carotene, |
| 12 | mcg Vitamin B₁₂, |
| 10 | mg Vitamin B₆, |
| 20 | mg Vitamin B₃, |
| 3 | mg Vitamin B₂, |
| 4 | mg Vitamin B₁, |
| 30 | I. U. Vitamin E, |
| 40 | mg Iron, |
| 22 | mg Zinc, |
| 120 | mg Vitamin C |
| 30 | mcg Molybdenum, |
| 80 | mcg Chromium, |
| 4,000 | I.U. Vitamin A, |
| 60 | mg Potassium, |
| 12 | mg Pantothenic Acid, |
| 0.8 | mg Folic Acid, |
| 240 | mcg Biotin, |
| 2 | mg Copper, |
| 150 | mcg Iodine, |
| 200 | mg Magnesium, |
| 60 | mcg Selenium, and |
| 320 | mg Phosphorous, |

### EXAMPLE 3

### Common Cold Discomfort Reliever and Calcium

30 mg pseudoephedrine HCl (nasal decongestant), 200 mg guaifenesin (expectorant), 15 mg detromethorphan HBr (cough suppressant) and 200 mg calcium carbonate are mixed to form a product mixture.

### EXAMPLE 4

The following components in the amounts indicated are mixed together to form a product mixture.

| | |
|---|---|
| 200 | mg ibuprofen, |
| 380 | mg Calcium, |
| 400 | I.U. Vitamin D, |
| 600 | I.U. Beta-carotene, |
| 12 | mcg Vitamin B₁₂, |
| 10 | mg Vitamin B₆, |
| 20 | mg Vitamin B₃, |
| 3 | mg Vitamin B₂, |
| 4 | mg Vitamin B₁, |
| 30 | I. U. Vitamin E, |
| 40 | mg Iron, |
| 22 | mg Zinc, |
| 120 | mg Vitamin C, |
| 30 | mcg Molybdenum, |
| 80 | mcg Chromium, |
| 4,000 | I.U. Vitamin A, |
| 60 | mg Potassium, |
| 12 | mg Pantothenic Acid, |
| 0.8 | mg Folic Acid, |
| 240 | mcg Biotin |
| 2 | mg Copper, |
| 150 | mcg Iodine, |
| 200 | mg Magnesium, |
| 60 | mcg Selenium, and |
| 320 | mg Phosphorous, |

In Examples 5A-5C ibuprofen is added but otherwise they follow Example 1 of U.S. Pat. No. 5,869,084, which is incorporated herein by reference in its entirety.

### EXAMPLE 5A

Each component of following compositions in Table I in column under lactating in the amount of the midpoint of the range is mixed to form a product mixture.

### EXAMPLE 5B

Each component of following compositions in Table I in column under non-lactating in the amount of the midpoint of the range is mixed to form a product mixture.

### EXAMPLE 5C

Each component of following compositions in Table I in column under menopausal in the amount of the midpoint of the range is mixed to form a product mixture.

TABLE I

| | Example 5A | Example 5B | Example 5C |
|---|---|---|---|
| Component | Lactating | Non-Lactating | Menopausal |
| Ibuprofen mg | 50-100 | 50-300 | 50-300 |
| Calcium, mg | 320-480 | 160-240 | 320-480 |
| Vitamin D, I.U. | 400-600 | 320-480 | 320-480 |
| Beta-carotene, I.U. | 400-1200 | 250-750 | 250-750 |
| Vitamin B₁₂, mcg | 9.6-14. | 9.6-14.4 | 20-30 |
| Vitamin B₆, mg | 8-12 | 8-12 | 2.4-3.6 |
| Vitamin B₃, mg | 20-30 | 20-30 | 16-24 |
| Vitamin B₂, mg | 2.7-4.0 | 2.7-4.0 | 1.3-2.0 |
| Vitamin B₁, mg | 3.2-4.8 | 3.2-4.8 | 1.2-1.8 |
| Vitamin E, I.U. | 24-36 | 24-36 | 70-110 |
| Iron, mg | 28-43 | 39-42 | 7-11 |
| Zinc, mg | 20-30 | 20-30 | 16-24 |
| Vitamin C, mg | 95-145 | 95-175 | 190-300 |
| Molybdenum, mcg | 20-30 | 40-60 | 40-60 |
| Chromium, mcg | 40-60 | 80-120 | 80-120 |
| Vitamin A, I.U. | 3,600-10,000 | 3,600-5,400 | 3,600-5,400 |
| Potassium, mg. | 40-60 | 40-60 | 64-96 |
| Pantothenic Acid, mg | 12-18 | 8-12 | 8-12 |
| Folic Acid, mg | 0.8-1. | 0.4-0.8 | 0.4-0.6 |
| Biotin, mcg | 40-60 | 240-360 | 240-360 |
| Copper, mg | 1.6-2.4 | 1.6-2.4 | 1.6-2.4 |
| Iodine, mcg | 120-180 | 120-180 | 120-180 |
| Magnesium, mg | 160-240 | 160-240 | 160-240 |
| Selenium, mcg | 50-70 | 50-70 | 50-70 |
| Phosphorous, mg | 320-480 | 160-240 | 320-480 |

### EXAMPLE 6

100 mg pseudophedrine hydrochloride, 50 mg dextromethorphan hydrobromide, 80 mg acetaminophen and 100 mg vitamin C are mixed to form a product mixture.

### EXAMPLE 7

200 mg of ibuprofen, 30 mg pseudoephedrine HCl and 100 mg vitamin C are mixed to form a product mixture.

### EXAMPLE 8

100 mg vitamin C and 200 mg of aspirin (acetylsalicylic acid) are mixed to form a product mixture.

Table II summarizes the active ingredients of Examples 1-8 above.

TABLE II

| Example | NUTRITIONAL | |
|---|---|---|
| Number | SUPPLEMENT | DISCOMFORT RELIEVER |
| 1 | VITAMIN C | IBUPROFEN |
| 2 | MULTIPLE | IBUPROFEN |
| | VITAMINS & | |
| | MINERALS | |
| 3 | CALCIUM | pseudoephedrine HCl |
| | | guaifenesin & detromethorphan HBr |
| 4 | MULTIPLE | IBUPROFEN |
| | VITAMINS & | |
| | MINERALS | |
| 5A, 5B | MULTIPLE | IBUPROFEN |
| and 5C | VITAMINS & | |
| | MINERALS | |
| 6 | VITAMIN C | pseudophedrine hydrochloride, |
| | | dextromethorphan hydrobromide, |
| | | acetaminophen, |
| 7 | VITAMIN C | ibuprofen, pseudoephedrine HCl |
| 8 | VITAMIN C | ASPIRIN |

In Examples 9-14 a first container encloses a first set of tablets and has a first label which indicates that the tablets therein are for relieving discomfort and supplementing nutrition. The first label also indicates the amount of each nutritional supplement and each discomfort reliever per tablet enclosed in the first container. A second container encloses a second set of tablets and has a second label which indicates that the tablets therein are for relieving discomfort and supplementing nutrition. The second label also indicates the amount of each nutritional supplement and each discomfort reliever per tablet enclosed in the second container. The first and second set of tablets have the active ingredients shown in the following table III. Each of the tablets of Examples 9-14 is made by blending a portion of powder having the indicated amount of the discomfort reliever and a portion of powder having the indicated amount of the nutritional supplement. Each blend is pressed into a tablet. The first and second containers are supported on a display shelf.

The tablets of set 1 of Example 9 are formed by blending the powder formed by grinding one of these 200 mg ibuprofen tablets with 33.33 percent of the powder formed by grinding a 600 mg Calcium tablet. The tablets of set 2 of Example 9 are formed by blending the powder formed by grinding one of these 200 mg ibuprofen tablets with 66.66 percent of the powder formed by grinding a 600 mg Calcium tablet. A 200 mg of ibuprofen tablet is sold by Whitehall Robins Healthcare trademark Advil (pain reliever). A 600 mg Calcium tablet is sold by The Fleming Company Inc.

The tablets of set 1 of Example 10 are formed by blending the powder formed by grinding a 200 mg ibuprofen tablet with 40 percent of the powder formed by grinding a 500 mg vitamin C tablet. The tablets of set 2 of Example 10 are formed by blending the powder formed by grinding a 200 mg ibuprofen tablet with 80 percent of the powder formed by grinding a 500 mg vitamin C tablet. A 500 mg vitamin C tablet is sold by Leiner Health Products Inc under the trademark YOUR LIFE Immune System.

The tablets of set 1 of Example 11 are formed by blending the powder formed by grinding a 200 mg ibuprofen tablet with 50 percent of the powder formed by grinding a multiple vitamin and mineral tablet having the composition shown in Table IV. The tablets of set 2 of Example 11 are formed by blending the powder formed by grinding a 200 mg ibuprofen tablet with 100 percent of the powder formed by grinding a multiple vitamin and mineral tablet having the composition shown in Table IV. A commercially available source multiple vitamin and mineral tablet is sold by Bayer Corporation under the trademark One A Day Essential and has the composition shown in Table IV. Another commercially available multiple vitamin and mineral tablet is sold by Whitehall Robins Healthcare, sold under the trademark Centrum Silver (with lutein iron free formula) and has the composition shown in Table III.

The tablets of set 1 of Example 12 are formed by blending the powder formed by grinding a 200 mg ibuprofen tablet with 40 percent of the powder formed by grinding a 500 mg vitamin C tablet. The tablets of set 2 of Example 12 are formed by blending the powder formed by grinding a 200 mg ibuprofen tablet with 33.33 percent of the powder formed by grinding a 600 mg Calcium tablet. A 500 mg vitamin C tablet is sold by Leiner Health Products Inc the trademark YOUR LIFE Immune System. A 600 mg Calcium tablet is sold by The Fleming Company Inc.

The tablets of set 1 of Example 13 are formed by blending the powder formed by grinding a 200 mg ibuprofen tablet with 20 percent of the powder formed by grinding a 500 mg vitamin C tablet. The tablets of set 2 of Example 13 are formed by blending the powder formed by grinding a 200 mg ibuprofen tablets with 40 percent of the powder formed by grinding a 500 mg vitamin C tablet. A 500 mg vitamin C tablet is sold by Leiner Health Products Inc the trademark YOUR LIFE Immune System.

The tablets of set 1 of Example 14 are formed by blending the powder formed by grinding a tablet containing 250 mg acetaminophen, 250 mg aspirin and 65 mg caffeine with 50 percent of the powder formed by grinding a multiple vitamin and mineral tablet having the composition shown in Table IV. The tablets of set 2 of Example 14 are formed by blending the powder formed by grinding a tablet containing 250 mg acetaminophen, 250 mg aspirin and 65 mg caffeine with 100 percent of the powder formed by grinding a multiple vitamin and mineral tablet having the composition shown in Table IV. A tablet containing 250 mg acetaminophen, 250 mg aspirin and 65 mg caffeine is sold by Bristol Myers Squibb Co. under the trademark of Excedrin Migraine (pain reliever). A commercially available source multiple vitamin and mineral tablet is sold by Bayer Corporation under the trademark One A Day Essential and has the composition shown in Table IV. Another commercially available multiple vitamin and mineral tablet is sold by Whitehall Robins Healthcare, sold under the trademark Centrum Silver (with lutein iron free formula) and has the composition shown in Table III.

Table III shows has the composition of nutritional ingredients of a single tablet commercially available from Whitehall Robins Healthcare, sold under the trademark Centrum Silver (with lutein iron free formula) multiple vitamins & minerals.

TABLE III

| | |
|---|---|
| 5,000 | I.U. Vitamin A |
| | (20% as Beta- |
| | carotene), |
| 60 | mg Vitamin C, |
| 400 | I.U.Vitamin D, |
| 45 | I.U.Vitamin E, |
| 10 | mcg Vitamin K, |
| 1.5 | mg Thiamin, |
| 1.7 | mg Riboflavin, |
| 20 | mg Niacin, |
| 3 | mg Vitamin B₆, |
| 400 | mcg Folic Acid, |
| 25 | mcg Vitamin B₁₂, |
| 30 | mcg Biotin |
| 10 | mg Pantothenic |
| | Acid, |
| 200 | mg Calcium and |
| 48 | mg Phosphorous, |
| 150 | mcg lodine, |
| 100 | mg Magnesium, |
| 15 | mg Zinc, |
| 20 | mcg Selenium, |
| 2 | mg Copper, |
| 2 | mg Manganese, |
| 150 | mcg Chromium, |
| 75 | mcg Molybdenum, |
| 72 | mg Chlorine |
| 80 | mg Potassium, |
| 150 | mcg Boron, |
| 5 | mcg Nickelm, |
| 2 | mg Silicon, |
| 10 | mcg Vanadium, and |
| 250 | mcg Lutein. |

Alternatively, the composition shown in Table IV is used as the multiple vitamins and minerals of nutritional supplement (NS) in Examples 11, 12 and 14 A single tablet having this composition is commercially available from Whitehall Robins Healthcare under the trademark Centrum Silver (with lutein iron free formula), or from Bayer Corporation under the trademark One A Day Essential multiple vitamin & mineral tablets.

### Table IV

5,000 I.U. Vitamin A 1.5 mg Vitamin B₁, 400 mcg Folic Acid,
60 mg Vitamin C, 1.7 mg Vitamin B₂, 6 mcg Vitamin B₁₂ and
400 I.U. Vitamin D, 20 mg Niacin, 10 mg Pantothenic
30 I.U. Vitamin E, 2 mg Vitamin B₆, Acid,

Examples 9-14 are summarized in the following table V:

TABLE V

| | | | NUTRITIONAL | | DISCOMFORT |
|---|---|---|---|---|---|
| | | amount | SUPPLEMENT | | RELIEVER |
| EXAMPLE | SET | of NS | (NS) | mg of DR | (DR) |
| 9 | 1 | 200 mg | CALCUIM | 200 | IBUPROFEN |
| | 2 | 400 mg | CALCUIM | 200 | IBUPROFEN |
| 10 | 1 | 200 mg | VITAMIN C | 200 | IBUPROFEN |
| | 2 | 400 mg | VITAMIN C | 200 | IBUPROFEN |
| 11 | 1 | 50% of | MULTIPLE | 200 | IBUPROFEN |
| | | source | VITAMINS & | | |
| | | tablet | MINERALS | | |
| | 2 | 100% of | MULTIPLE | 200 | IBUPROFEN |
| | | source | VITAMINS & | | |
| | | tablet | MINERALS | | |
| 12 | 1 | 200 mg | CALCUIM | 200 | IBUPROFEN |
| | 2 | 200 mg | VITAMIN C | 200 | IBUPROFEN |
| 13 | 1 | 100 mg | VITAMIN C | 200 | IBUPROFEN |
| | 2 | 200 mg | VITAMIN C | 200 | IBUPROFEN |
| 14 | 1 | 50% of | MULTIPLE | 250 | ASCETAMINOPHEN |
| | | source | VITAMINS & | 250 | ASPIRIN & |
| | | tablet | MINERALS | 65 | CAFFEINE |
| | 2 | 100% f | MULTIPLE | 250 | ASCETAMINOPHEN |
| | | source | VITAMINS & | 250 | ASPIRIN & |
| | | tablet | MINERALS | 65 | CAFFEINE |

In each of Examples 15-23 a nutritional supplement source tablet and a discomfort reliever source tablet, having the active ingredients listed in the table VI below, are crushed into a fine powder, mixed together and pressed into a combined tablet by tablet making procedures known in the art. Each source tablet is commercially available. The combined tablet is enclosed in a combined product container having affixed thereto the label of the container of the discomfort reliever source tablets. Also affixed to the combined product container are the portions of the label of the container of the nutritional supplement source tablets identifying the nutritional ingredients and the inactive ingredients of the nutritional supplement source tablets. Thus, each combined product container has labeling identifying the discomfort reliever, vitamins and minerals and the amounts thereof contained in the enclosed combined tablet. For each combined product container the portion of the label specifying discomforts on the discomfort reliever source container is removed and affixed to the container for the combined tablet.

TABLE VI

| | COMBINED | NUTRITIONAL | DISCOMFORT |
|---|---|---|---|
| | TABLET | SUPPLEMENT | RELIEVER SOURCE |
| EXAMPLE | COMPOSITION | SOURCE TABLET | TABLET |
| 15 | the nutritionally | nutritionally effective | 250 mg acetaminophen, |
| | effective components | components in the | 250 mg aspirin and 65 mg |
| | in the amounts listed in | amounts listed in Table | caffeine sold by |
| | Table IV and 250 mg | IV sold by Bayer Corp. | Bristol Myers Squibb Co |
| | acetaminophen, 250 mg | under trademark: | under trademark |
| | aspirin and 65 mg | ONE A DAY | Excedrin Migraine (pain |
| | caffeine | ESSENTIAL | reliever tablets) |
| 16 | the nutritionally | nutritionally effective | 200 mg naproxin sold by |
| | effective components | components in the | Bayer Corp. under |
| | in the amounts listed in | amounts listed in Table | trademark: Aleve |
| | Table IV and 200 mg | IV sold by Bayer Corp. | (pain reliever tablets) |
| | naproxin | under trademark: | |
| | | ONE A DAY | |
| | | ESSENTIAL | |
| 17 | the nutritionally | nutritionally effective | 325 mg aspirin sold by |
| | effective components | components in the | Bayer Corp. sold under |
| | in the amounts listed in | amounts listed in Table | trademark Bayer aspirin |
| | Table IV and 325 mg | IV sold by Bayer Corp. | (pain reliever tablets) |
| | aspirin | under trademark: | |
| | | ONE A DAY | |
| | | ESSENTIAL | |
| 18 | the nutritionally | ingredients in the | 200 mg of ibuprofen sold |
| | effective components | amounts listed in Table | by Whitehall Robins |
| | in the amounts listed in | III sold by Whitehall | Healthcare sold under |
| | Table III and 200 mg of | Robins Healthcare sold | trademark |
| | ibuprofen | under trademark | ADVIL (pain reliever |
| | | CENTRUM SILVER | tablets) |
| | | (with lutein) | |
| 19 | 500 mg vitamin C | 500 mg vitamin C sold | 200 mg caffeine sold by |
| | and200 mg caffeine | by Leiner Health | Bristol Myers Squibb Co. |
| | | Products Inc. under | under trademark NO |
| | | trademark | DOZ |
| | | YOUR LIFE Immune | |
| | | System tablets | |
| 20 | 500 mg vitamin C and | 500 mg vitamin C sold | 25 mg oxyamine |
| | 25 mg oxyamine | by Leiner Health | succinate sold by Pfizer |
| | succinate | Products Inc. under | sold under trademark |
| | | trademark | Unison Sleep Tabs |
| | | YOUR LIFE Immune | |
| | | System tablets | |
| 21 | 500 mg vitamin C, 12.5 mg | 500 mg vitamin C sold | 12.5 mg |
| | diphenhydramine | by Leiner Health | diphenhydramine HCl, 30 mg |
| | HCl, 30 mg | Products Inc. under | pseudoephedrine |
| | pseudoephedrine HCl, | trademark | HCl, 500 mg |
| | and 500 mg | YOUR LIFE Immune | acetaminophen sold by |
| | acetaminophen | System tablets | Park Davis under |
| | | | trademark BENADRYL |
| | | | Allergy/sinus/headache |
| 22 | 500 mg vitamin C, 30 mg | 500 mg vitamin C sold | 30 mg pseudoephedrine |
| | pseudoephedrine | by Leiner Health | HCl, 500 mg |
| | HCl, and 500 mg | Products Inc. under | acetaminophen sold by |
| | acetaminophen | trademark | Warner Lambert under |
| | | YOUR LIFE Immune | trademark |
| | | System tablets | Sudafed Allergy caplets |
| 23 | 500 mg vitamin C, 30 mg | 500 mg vitamin C sold | 30 mg pseudoephedrine |
| | pseudoephedrine | by Leiner Health | HCl, 500 acetaminophen |
| | HCl, 500 mg | Products Inc. under | sold by Smith Kline |
| | acetaminophen | trademark | Beecham under |
| | | YOUR LIFE Immune | trademark |
| | | System tablets | CONTAC cold caplets |

Trademarks of Bristol Myers Squibb Co. include Theragran Heart Right (multiple vitamin and mineral tablets), Excedrin Migraine (pain reliever tablets: containing 250 mg acetaminophen, 250 mg aspirin and 65 mg caffeine: as the active ingredients) and NO DOZ containing 200 mg caffeine. Trademarks of Whitehall Robins Healthcare include Centrum Silver tablets containing: multiple vitamins and minerals, Advil (pain reliever tablets containing: 200 mg of ibuprofen per tablet as its only active ingredient) and Anacin (pain reliever tablets). Trademarks of Bayer Corporation include One A Day Essential (multiple vitamin and mineral tablets) and Bayer aspirin (pain reliever tablets containing: 325 mg aspirin as its only active ingredient) and Aleve (pain reliever tablets containing: 200 mg naproxin as its only active ingredient). Trademarks of Leiner Health Products Inc include YOUR LIFE Immune System tablets (vitamin C 500 mg). Trademarks of Park Davis include BENADRYL Allergy/sinus/headache caplets containing 12.5 mg diphenhydramine HCl, 30 mg pseudoephedrine HCl, 500 acetaminophen). Trademarks of Warner Lambert include Sudafed Allergy caplets (30 mg pseudoephedrine HCl, 500 acetaminophen). Trademarks of Pfizer include Unison Sleep Tabs (25 mg oxyamine succinate). Trademarks of Smith Kline Beecham include CONTAC cold caplets (30 mg pseudoephedrine HCl, 500 acetaminophen). See also U.S. Pat. No. 5,895,663 incorporated herein by reference in its entirety.

Fewer containers and fewer tablets are required by the invention to relieve discomfort and supplement nutrition, than are required by prior art discomfort relievers and prior art nutritional supplements. Twice as many containers and tablets are required by the prior art as are required by the invention to provide the same benefits. For example a consumer having continuing discomforts may have a set of six containers at each of three locations: at home, at an office and in a hand bag (or attache case). Each set of containers includes three containers of discomfort relievers: allergy, headache pain and cold pain, and three containers of nutritional supplements: multiple vitamin and mineral, vitamin C and calcium. Combined discomfort reliever and nutritional supplement of the invention requires only three containers for the same discomfort relief and more consistent nutritional supplement.

This is a total reduction of nine containers of tablets (or fifty percent savings). The consumer has the convenience of nine (fifty percent) fewer containers to purchase, carry and store and fifty percent fewer tablets to consume while maintaining the availability of active ingredients and increasing the likelihood of consuming nutritional supplements.

More specifically, six containers of tablets are replaced by three containers of tablets, for an overall reduction of three containers of tablets. Headache pain reliever and vitamin C tablets containing ibuprofen and vitamin C are formed for example by following Example 1. Allergy discomfort reliever and multiple vitamin and mineral nutritional supplement tablets containing diphenylhydramine HCl and multiple vitamins and minerals are formed by following Example 2. Cold discomfort reliever and calcium nutritional supplement tablets containing pseudoephedrine HCl (nasal decongestant), and calcium nutritional supplement are formed by following Example 3.

Benefits of the invention are available for example to a consumer having continuing discomfort having a container of headache pain reliever and vitamin C tablets containing ibuprofen and vitamin C formed by following Example 1. For headache pain relief the consumer orally consumes one tablet every 4 hours. Over each 24 hour period the consumer consumes 600 mg of vitamin C without having to store, restock or swallow large (500 mg or larger) tablets.

It should be understood that while the present invention has been described in considerable detail with respect to certain specific embodiments thereof, it should not be considered limited to such embodiments but may be used in other ways without departure from the spirit and the scope of the invention.

1-20. (canceled)

21. A method of indication for supplementing nutrition and for relief of discomfort, comprising: enclosing a unit dose of orally consumable material in an enclosure having indications and a twist off cap,
said unit dose comprising a predetermined pharmaceutically effective amount from 50 mg to 300 mg of a discomfort reliever consisting essentially of aspirin, and a predetermined nutritionally effective amount from 50 mg to 1000 mg of a nutritional supplement consisting essentially of calcium,
said indications indicating said pharmaceutically effective amount of said discomfort reliever in said unit dose, said indications indicating said nutritionally effective amount of said nutritional supplement in said unit dose,
said indications indicating between one and fifty percent of a daily value for said nutritional supplement in said unit dose,
said indications indicating said unit dose as being for women,
said indications indicating an expiration date for said unit dose and said indications indicating said discomfort reliever in said unit dose as being for relief of at least one discomfort selected from the group consisting of aches, pain and fever.

22. The method of claim 21 wherein said indications indicate instructions for consuming the unit dose for supplementing nutrition, and said indications indicate said discomfort reliever in said unit dose as being for relief of aches and pain.

23. The method of claim 21 wherein said discomfort reliever consists of aspirin, and said nutritional supplement consists of calcium adapted of supplementing nutrition.

24. The method of claim 21 wherein said discomfort reliever is a nonprescription pain reliever.

25. A method of indication for supplementing nutrition and for relief of discomfort, comprising: enclosing a unit dose of orally consumable material in an enclosure having indications,
said unit dose comprising a predetermined pharmaceutically effective amount of a discomfort reliever consisting essentially of aspirin and a predetermined nutritionally effective amount of a nutritional supplement consisting essentially of calcium,
said indications indicating said pharmaceutically effective amount of said discomfort reliever in said unit dose, said indications indicating said nutritionally effective amount of said nutritional supplement in said unit dose,
said indications indicating said nutritional supplement for supplementing nutrition, and
said indications indicating said discomfort reliever in said unit dose as being for relief of at least one discomfort selected from the group consisting of aches, pain and fever.

26. The method of claim 25 wherein said discomfort reliever consists essentially of from 50 mg to 300 mg of aspirin said nutritional supplement consists essentially of from 50 mg to 1000 mg of calcium.

27. The method of claim 25 further comprising storing said unit dose, shipping said unit dose, and consuming said predetermined nutritionally effective amount of calcium and said predetermined pharmaceutically effective amount of aspirin in said unit dose to supplement nutrition and relieve said discomfort.

28. The method of claim 25 wherein said nutritional supplement is from 50 mg to 1200 mg of calcium and said indications indicate said unit dose is for women.

29. The method of claim 25 wherein said indications indicate an expiration date for said unit dose.

30. The method of claim 25 wherein said enclosure is a plastic bottle having a twist off cap.

31. The method of claim 25 wherein said indications indicate a daily value for said nutritional supplement in said unit dose, and said indications indicate instructions for consuming said unit dose for supplementing nutrition.

32. The method of claim 25 wherein said indications indicate between one and fifty percent of a daily value for said nutritional supplement in said unit dose.

33. A method of indication for supplementing nutrition and for relief of discomfort, comprising: enclosing a unit dose of orally consumable material in an enclosure having indications,
said unit dose comprising a predetermined pharmaceutically effective amount from about 50 mg to about 300 mg of a discomfort reliever consisting essentially of aspirin and a predetermined nutritionally effective amount from about 50 mg to about 1000 mg of a nutritional supplement consisting essentially of calcium,
said indications indicating said pharmaceutically effective amount of said discomfort reliever in said unit dose, said indications indicating said nutritionally effective amount of said nutritional supplement in said unit dose,
said indications indicating a percent of a daily value for said nutritional supplement in said unit dose, and
said indications indicating said discomfort reliever in said unit dose as being for relief of at least one discomfort selected from the group consisting of aches, pain, and fever.

34. The method of claim 33 wherein said enclosure is a plastic bottle having a twist off cap.

35. The method of claim 33 wherein said indications indicate said unit dose is for women.

36. The method of claim 33 further comprising shipping said unit dose, and consuming said predetermined nutritionally effective amount of calcium and said predetermined pharmaceutically effective amount of aspirin in said unit dose.

37. The method of claim 33 wherein said indications indicate instructions for consuming the unit dose for supplementing nutrition.

38. The method of claim 33 wherein said discomfort reliever consists of aspirin, and said nutritional supplement consists of calcium adapted of supplementing nutrition.

39. The method of claim 33 wherein said indications indicate an expiration date for said unit dose and said indications indicate a bar code identifying said unit dose.

40. The method of claim 33 wherein said indications indicate between one and fifty percent of a daily value for said nutritional supplement in said unit dose.
